(19) United States
(12) Patent Application Publication
(10) Pub. No.: US 2015/0125851 A1
(43) Pub. Date: May 7, 2015

(54) OPTICAL SENSOR AND MANUFACTURING METHOD THEREOF, AND DETECTION METHOD UTILIZING SAME

(71) Applicant: Panasonic Intellectual Property Management Co., Ltd., Osaka (JP)

(72) Inventors: Kiyoshi Hashimotodani, Kyoto (JP); Yusuke Kitagawa, Kyoto (JP)

(21) Appl. No.: 14/397,469

(22) PCT Filed: Apr. 8, 2013
(86) PCT No.: PCT/JP2013/002387

§ 371 Date: Oct. 27, 2014

Related U.S. Application Data
(60) Provisional application No. 61/639,320, filed on Apr. 27, 2012.

(51) Int. Cl. G01N 33/569; G01N 21/552

(52) U.S. Cl. CPC G01N 33/56983; G01N 21/553; G01N 2201/061
USPC 435/5; 435/288.7

(57) ABSTRACT

An optical sensor is configured to be used with trappers specifically bound to an object substance to detect whether the object substance exists or not in a specimen. The optical sensor includes a first metal layer made of gold having a lower surface and an upper surface which is configured to have an electromagnetic wave supplied thereto, and a second layer made of gold having an upper surface facing the lower surface of the first metal layer. A hollow area configured to be filled with the specimen is provided between the first metal layer and the second metal layer. The trappers are physically bonded to at least one of a lower side of the first metal layer and an upper side of the second metal layer. The thickness of the first metal layer is not smaller than 5 nm and not larger than 30 nm. The optical sensor has a small size and a simple structure.

### TECHNICAL FIELD

This invention relates to an optical sensor utilizing an optical interference phenomenon, to be used for detecting, e.g. a virus.

### BACKGROUND ART

FIG. 10 is a cross-sectional view of conventional optical sensor 100 disclosed in PTL 1. Optical sensor 100 includes prism 101, metal layer 102 disposed on a lower surface of prism 101, insulation layer 103 fixed to a lower surface of metal layer 102, and trappers 104 fixed to a lower surface of insulation layer 103. Trapper 104 is made of, e.g. an antibody.

A surface plasmon wave, a compressional electron wave, (not shown) exists at an interface between metal layer 102 and insulation layer 103. Light source 105 is placed above prism 101. A P-polarized light is emitted from light source 105 and enters to prism 101 at a total reflection condition. At this moment, an evanescent wave is produced on a surface of metal layer 102 and a surface of insulation layer 103. The light totally reflected by metal layer 102 is received by detector 106 to detect an intensity of the light.

If the wave number of the evanescent wave is identical to that of the surface plasmon wave to satisfy a wave-number matching condition, energy of the light supplied from light source 105 is used for exciting the surface plasmon wave, accordingly decreasing an intensity of the reflected light. The wave-number matching condition depends on an incident angle of the light supplied by light source 105. Therefore, when detector 106 detects the intensity of the reflected light while changing the incident angle, the detector determines that the intensity of the reflected light decreases at a certain incidence angle.

A resonance angle at which the intensity of the reflected light becomes a minimum depends on a dielectric constant of insulation layer 103. When a specific binding substance including an analyte, an object substance in a specimen, and trapper 104 which are specifically bound is formed on a lower surface of insulation layer 103, the dielectric constant of insulation layer 103 changes accordingly. Therefore, by monitoring the change in the resonance angle, a bonding strength and a speed of the specific binding between the analyte and trapper 104 are monitored.

However, optical sensor 100 includes light source 105 supplying the P-polarized light and prism 101 on an upper surface of metal layer 102, hence having a large size and a complicated structure.

PTL 2 discloses another conventional optical sensor which has a small size and a simple structure.

FIG. 11 is a schematic view of conventional optical sensor 201 disclosed in PTL 2. Optical sensor 201 includes first metal layer 202 and second metal layer 203 having an upper surface facing a lower surface of the first metal layer. First metal layer 202 has a thickness ranging from 30 nm to 45 nm. Second metal layer 203 has a thickness not smaller than 100 nm. Hollow area 204 is provided between first metal layer 202 and second metal layer 203. Hollow area 204 is configured to be filled with specimen 208 containing solutes 208A, 208B and 208C. Trappers 202 is physically bonded to at least one of a lower side of first metal layer 202 and an upper side of second metal layer 203.

A light supplied from light source 209, an electromagnetic wave source, to first metal layer 202 causes an optical resonance at first interface 202B between first metal layer 202 and hollow area 204 and at second interface 203B between second metal layer 203 and hollow area 204. If solute 208C which is an object substance (an analyte) to be specifically bound to trapper 207 is included in specimen 208, trapper 207 are specifically bound to the analyte and changes a dielectric constant in the hollow area. This changes a condition for causing the optical resonance, and changes a resonance absorption wavelength for the light supplied from light source 209. This change can be visually detected as a change in color.

Optical sensor 201 does not require a prism. The light supplied from light source 209 is not required to be specifically polarized or to have a specific coherence characteristic, hence providing optical sensor 201 with a small size and a simple structure.

### CITATION LIST

### Patent Literature

PTL 1: Japanese Patent Laid-Open Publication No. 2005-181296

PTL 2: International Publication WO2010/122776

### SUMMARY

An optical sensor is configured to be used with a plurality of trappers specifically bound to an object substance to detect whether the object substance exists or not in a specimen. The optical sensor includes a first metal layer made of gold having a lower surface and an upper surface which is configured to have an electromagnetic wave supplied thereto, and a second layer made of gold having an upper surface facing the lower surface of the first metal layer. A hollow area configured to be filled with the specimen is provided between the first metal layer and the second metal layer. The trappers are physically bonded to at least one of a lower side of the first metal layer and an upper side of the second metal layer. A thickness of the first metal layer is not smaller than 5 nm and not larger than 30 nm.

The optical sensor has a small size and a simple structure.

### BRIEF DESCRIPTION OF DRAWINGS

FIG. 1 is a cross-sectional view of an optical sensor according to an exemplary embodiment.

FIG. 2A is a schematic view of the optical sensor according to the embodiment for illustrating a trapper used in the optical sensor.

FIG. 2B schematically shows a specific binding of the trapper and an analyte according to the embodiment.

FIG. 3A schematically shows an aggregation of the trapper of the optical sensor according to the embodiment.

FIG. 3B schematically shows an aggregation of the trapper of the optical sensor according to the embodiment.

FIG. 4A is a schematic view of the optical sensor according to the embodiment.

FIG. 4B is a schematic view of the optical sensor according to the embodiment.

FIG. 5A shows a change in a reflection spectrum of a comparative example of an optical sensor.

FIG. 5B shows a change in a reflection spectrum of the optical sensor according to the embodiment.

FIG. 6 shows a change in the reflection spectrum to a refractive index of the optical sensor according to the embodiment.

FIG. 7 shows a relation between a peak wavelength of a pseudo peak structure and a refractive index of the optical sensor according to the embodiment.

FIG. 8 shows a change in the reflection spectrum of the optical sensor according to the embodiment.

FIG. 9 shows a relation between the peak wavelength of the pseudo peak structure and a thickness of a hollow area of the optical sensor according to the embodiment.

FIG. 10 is a cross-sectional view of a conventional optical sensor.

FIG. 11 is a cross-sectional view of another conventional optical sensor.

### DETAIL DESCRIPTION OF PREFERRED EMBODIMENT

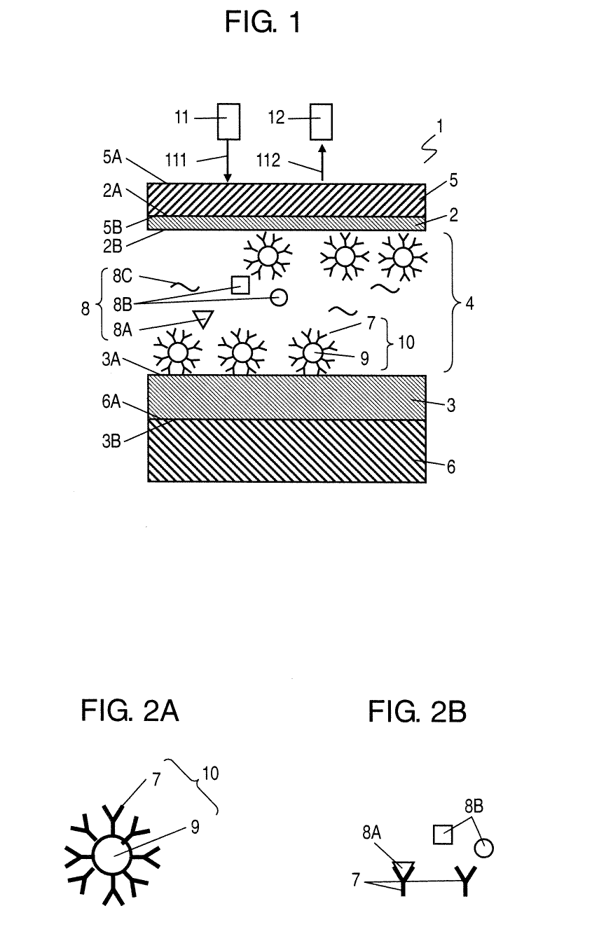

FIG. 1 is a schematic cross-sectional view of optical sensor 1 according to an exemplary embodiment of the invention. Optical sensor 1 includes metal layer 2 (a first metal layer), metal layer 3 (a second metal layer), and hollow area 4. Metal layer 2 has upper surface 2A and lower surface 2B, and is configured to have an electromagnetic wave supplied thereto. Metal layer 3 has upper surface 3A and lower surface 3B, and is configured to have an electromagnetic wave supplied thereto. Upper surface 3A of metal layer 3 faces lower surface 2B of metal layer 2. Metal layer 2 and metal layer 3 are made of gold. Hollow area 4 is provided between metal layers 2 and 3. Hollow area 4 is configured to be filled with specimen 8 containing a solute. Metal layer 2 has a thickness ranging from 5 nm to 30 nm. This configuration causes an optical resonance between metal layer 2 and metal layer 3 facing across the hollow area even if a light supplied to metal layer 2 is not P-polarized or a prism is not provided on upper surface 2A of metal layer 2. This arrangement provides optical sensor 1 with a small size and a simple structure.

Metal layer 2 has a thickness not smaller than 5 nm and not larger than 30 nm. This configuration moderates the optical resonance and increases a width of an absorption spectrum caused by the optical resonance.

Metal layer 2 and metal layer 3 are made of gold. This configuration merges an anomalous reflection of gold with the absorption spectrum caused by the optical resonance, and can provide a reflection spectrum having a pseudo peak structure. The reflection spectrum in the pseudo peak structure exhibits a pseudo single color of reflected light, and exhibits a sensitive change in color in response to a change in a resonance absorption wavelength of an optical resonance, thus increasing the sensitivity of optical sensor 1.

Holder 5 is fixed to upper surface 2A of metal layer 2 to securely maintain the shape of metal layer 2. Holder 5 is made of a material that can hardly attenuate incident electromagnetic wave 111 as to effectively supply incident electromagnetic wave 111 to metal layer 2.

Incident electromagnetic wave 111 is a visible light, an electromagnetic wave having a wavelength ranging from about 350 nm to 800 nm. Therefore, holder 5 is made of a transparent material, such as glass or transparent plastic material, which allows the visible light to pass efficiently. Holder 5 is preferably as thin as possible as long as it has an allowable mechanical strength.

Metal layer 3 has a thickness not smaller than 100 nm. If metal layer 3 has a thickness smaller than 100 nm, the electromagnetic wave supplied through metal layer 2 to hollow area 4 may partly leak out through metal layer 3. That is, energy of the electromagnetic wave to contribute to interference and to be utilized for detection may partly leak out of hollow area 4, hence reducing the sensitivity of optical sensor 1.

Lower surface 3B of metal layer 3 is fixed to upper surface 6A of holder 6 as to maintain the shape of metal layer 3.

Optical sensor 1 may include a spacer, such as a pillar or a wall, that holds metal layer 2 and metal layer 3 as to maintain a distance between metal layer 2 and metal layer 3. This configuration allows optical sensor 1 to hold hollow area 4 securely.

Trappers 7 are disposed inside hollow area 4. Trappers 7 are specifically bound to a specific object substance (analyte). The trapper may be an antibody, a receptor protein, an aptamer, a porphylin, or a high polymer formed by a molecular imprinting technology.

Trappers 7 may physically adhere to at least one of a lower side of lower surface 2B of metal layer 2 and an upper side of upper surface 3A of metal layer 3. Trappers 7 may not be disposed at at least the one of at the lower side of lower surface 2B of metal layer 2 and the upper side of upper surface 3A of metal layer 3.

FIG. 2A is a schematic view of composite body 10 used in optical sensor 1, and schematically shows disposition of trappers 7. As shown in FIG. 2A, trappers 7 are chemically bonded to a surface of particle 9 to form composite body 10. Composite body 10 is physically bonded to at least one of the lower side of lower surface 2B of metal layer 2 and the upper side of upper surface 3A of metal layer 3A, namely, composite body 10 is physically bonded to a surface of metal layer 2 or a surface of metal layer 3. However, when specimen 8 is injected from outside, composite body 10 is easily separated from the surface of metal layer 2 or metal layer 3 and re-dispersed into specimen 8.

Specimen 8 contains solvent 8C, analytes 8A dispersed in solvent 8C, and solutes 8B dispersed in solvent 8C. Analyte 8A is an object substance to be detected. Solute 8B is made of a material, such as protein, different from the material of analyte 8A. Solvent 8C is mainly made of water.

Particle 9 is made of polystyrene latex resin and has a diameter of, e.g. 100 nm. Trappers 7 may chemically adhere to particle 9 by a silane coupling reaction, or trapper 7 may be affixed to particle 9 through a self-assembled monolayer film.

FIG. 2B schematically shows a specific binding of trapper 7 and analyte 8A in optical sensor 1.

Trapper 7 is bound specifically only to analyte 8A. Namely, trapper 7 is bound to analyte 8A in specimen 8 but not to other solute 8B. This configuration selectively traps analyte 8A, a desired object substance to be detected, such as a virus antigen and a diagnostic protein marker.

Trapper 7 is affixed to particle 9. A large number of trappers 7 are fixed to particle 9. When composite bodies 10 are re-dispersed in specimen 8, trappers 7 easily contacts analyte 8A, hence effectively providing the specific binding between trapper 7 and analyte 8A.

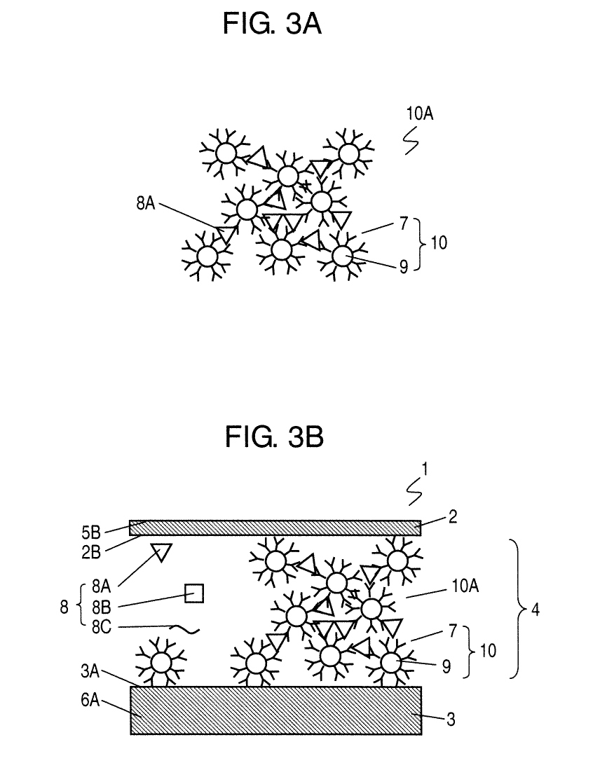

FIGS. 3A and 3B are schematic views of optical sensor 1 according to the embodiment for illustrating an aggregation of trappers 7 in optical sensor 1.

Analyte 8A ordinarily includes plural binding sites to be specifically bound to trapper 7. Trapper 7 on one particle 9 can be bound via analyte 8A to another trapper 7 affixed to another particle 9. That is, composite bodies 10 are bound to each other via analyte 8A, thereby forming aggregate 10A including composite bodies 10.

Polystyrene latex, a material of particle 9, has a refractive index of 1.59. In the case that solvent 8C of specimen 8 is made of water, solvent 8C has a refractive index of 1.3334.

When analyte 8A is contained in specimen 8, aggregate 10A of composite bodies 10 may be formed. Aggregate 10A fills at least a part of hollow area 4, and increases the refractive index of hollow area 4, hence changing a condition for causing an optical resonance in hollow area 4.

When analyte 8A is not contained in specimen 8, composite bodies 10 do not aggregate, and do not produce aggregate 10A, hence causing the refractive index of hollow area 4 to be equal to that of solvent 8C, i.e., water. To be precise, composite bodies 10 dispersed in the solvent cause refractive index in hollow area 4 to be slightly different from that of only water. However, influence of dispersed composite bodies 10 on the refractive index is practically negligible unless a density of composite bodies 10 is not as high as an emulsion state. As a result, the condition for causing an optical resonance in hollow area 4 is not changed. Therefore, if a change in the condition for causing the optical resonance is detected, it is determined whether analyte 8A exists or not.

Optical sensor 1 according to the embodiment can detect a change in a dielectric constant of a material suspended in hollow area 4. This configuration does not require that trappers 7 are chemically bonded to metal layer 2 or metal layer 3 via, e.g. a self-assembled monolayer (SAM), hence allowing optical sensor 1 to be manufactured by a simple process.

Particle 9 may be made of material other than popular polystyrene latex resin having a refractive index larger than water. For instance, particle 9 may be made of an inorganic material, such as metallic oxide, metal, or magnetic material, or an organic material, such as dendrimer.

In the case that particle 9 is made of a fine particle of titanium oxide, since the refractive index of titanium oxide is as large as at least 2.5, an amount of the change of the resonance wavelength becomes large, and further enhancement of the sensitivity is expected.

In the case that particle 9 is made of magnetic material, trappers 7 may be stirred by applying a magnetic field from outside of optical sensor 1 after specimen 8 is input into hollow area 4. This operation can efficiently causes specific binding of trapper 7 and analyte 8A.

Dendrimer may unify the shape thereof. Particles 9 made of dendrimer may decrease variation in the shapes of particles 9, accordingly reduces variation in performance of optical sensor 1.

According to the embodiment, particle 9 is a bead having a spherical shape, but may have a cubic shape. Particles 9 having cubic shapes can increase a rate of aggregated particles 9 (composite bodies 10) filling hollow area 4 since the particles can aggregate more easily than spherical shapes. On calculation, by neglecting the sizes of trapper 7 and analyte 8A, the rate of filling can be 100%. Meanwhile, the particles having spherical shapes provide the rate of filling of maximum 74%.

According to the embodiment, particle 9 has a diameter of 100 nm, but is not limited to it. Particle 9 having a diameter smaller than a half of the thickness of hollow area 4 may generally be input into hollow area 4. Particle 9 having a diameter smaller than about 50 nm in diameter reduces a Mie scattering effect, and may be almost transparent for a visible light. Hence, even if particles 9 are not made of transparent material, particles 9 does not prevent visible light from propagating in hollow area 4.

In optical sensor 1 according to the embodiment, the optical resonance wavelength changes as the refractive index of hollow area 4 between metal layer 3 and metal layer 4 changes. The refractive index n and the dielectric constant ε has a relation of n=ε^{1/2}, and the change in the refractive index is thus equivalent to the change in the dielectric constant. Hence, it is not necessary to affix trapper 7 securely to metal layer 2 and metal layer 3 by, e.g. chemical absorption.

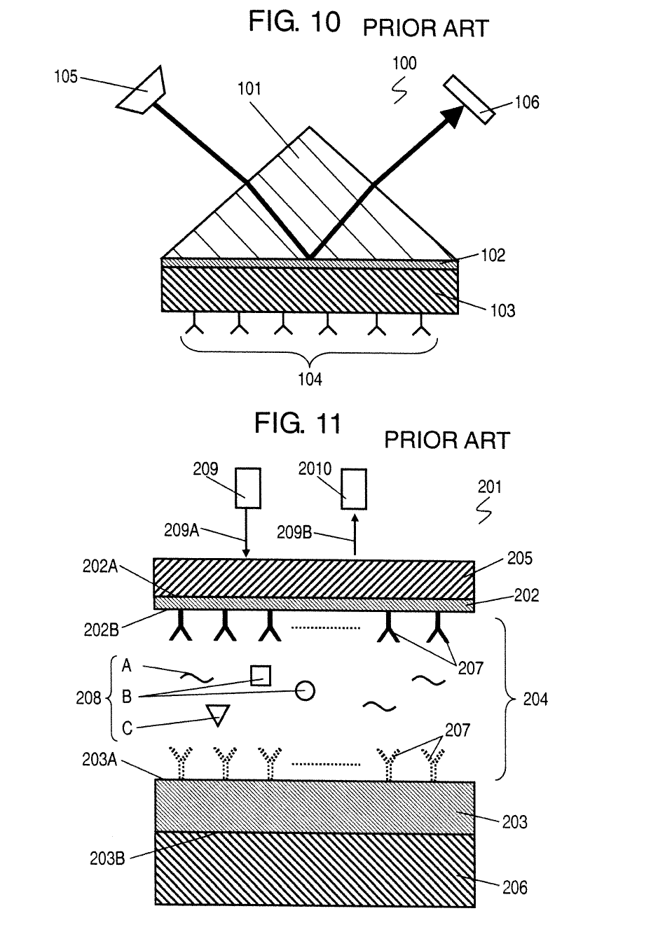

On the other hand, in conventional optical sensor 100 shown in FIG. 10, it is necessary to fix trapper 104 to the lower surface of insulation layer 103 by, e.g. chemical absorption for securing the sensitivity. Therefore, in processes for manufacturing optical sensor 1 according to the embodiment may have the disposition process of trapper 7, SAM film formation process for instance, omitted, thus enhancing manufacturing efficiency.

An operation of optical sensor 1 will be described below. According to the embodiment, electromagnetic source 11 is a light source, and incident electromagnetic wave 111 is a visible light.

Electromagnetic source 11 may be one of, e.g. a sun light, a halogen lamp and various discharge lamps, and preferably emits a white light containing components having wavelengths widely distributed. Electromagnetic source 11 does not include a device, such as a polarizing plate, for aligning polarization of light. Unlike conventional optical sensor 100 shown in FIG. 10, optical sensor 1 according to the embodiment can cause an optical resonance not only of a P-polarized light but also an S-polarized light or even a non-polarized light.

The wavelength of incident electromagnetic wave 111 causing the optical resonance may be controlled by adjusting at least one of an effective refractive index of hollow area 4 and a distance between metal layer 2 and metal layer 3.

The refractive index of trapper 7 on a surface of particle 9 does not practically contribute to the refractive index of composite body 10. An effective refractive index is determined by a distribution of the refractive index of specimen 8 input into hollow area 4 and the refractive index of particle 9 in composite body 10. Namely, the effective refractive index is an average refractive index in a space not smaller than the wavelength of incident electromagnetic wave 111 and reflected electromagnetic wave 112 on a propagation path thereof.

Detector 12 is provided above upper surface 2A of metal layer 2, and detects a visible light, reflected electromagnetic wave 112. Optical sensor 1 receives incident electromagnetic wave 111 supplied from light source 11, and then, detector 12 receives reflected electromagnetic wave 112 reflected by optical sensor 1. Detector 12 according to the embodiment is a visual inspection, but may be a light detector having a spectroscopic function.

In this configuration, incident electromagnetic wave 111, the light supplied from electromagnetic source 11, causes an optical resonance (interference) in hollow area 4. The wavelength causing the resonance is determined by the thickness of hollow area 4 and an effective refractive index of hollow area 4.

Holder 6 has a thickness preferably larger than that of holder 5. This arrangement increases mechanical strength of optical sensor 1, and prevents optical sensor 1 from deforming during its use and prevents a sensing characteristic thereof from deteriorating.

When hollow area 4 of optical sensor 1 is changed from a state shown in FIG. 1 to a state shown in FIG. 3B where hollow area 4 is filled with specimen 8 and composite bodies 10 is re-dispersed in specimen 8 to form aggregate 10A with analyte 8A, the resonance wavelength in the optical resonance of optical sensor 1 is changed. More specifically, as aggregate 10A is formed, distribution of the refractive index of composite body 10 (practically of particle 9) is changed, changing the effective refractive index of hollow area 4 between metal layer 2 and metal layer 4, thereby changing the resonance wavelength of the optical resonance of optical sensor 1.

A process for causing the optical resonance in optical sensor 1 according to the embodiment will be detailed below. Metal layer 2 has a thickness not larger than about 30 nm for passing incident electromagnetic wave 111 through metal layer 2. Upper surface 2A of metal layer 2 is fixed to lower surface 5B of holder 5 for maintaining the shape of metal layer 2. Similarly, metal layer 3 is fixed to upper surface 6A of holder 6 for maintaining the shape of metal layer 3.

Incident electromagnetic wave 111 in a visible light wavelength range enters to upper surface 2A of metal layer 2. Metal layer 2 is so thin, that incident electromagnetic wave 111 may pass through metal layer 2, propagates in hollow area 4, and reach metal layer 3.

Metal layer 3 preferably has a film thickness not smaller than 100 nm. The thickness smaller than 100 nm may allow electromagnetic wave 111 to pass through metal layer 3 and deteriorate the sensitivity of optical sensor 1.

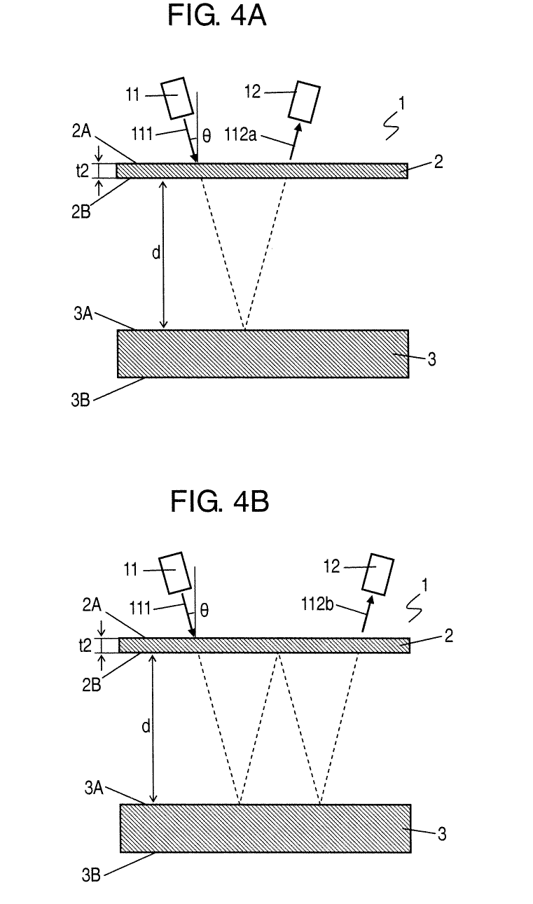

FIGS. 4A and 4B are schematic views of optical sensor 1. Incident electromagnetic wave 111 reflected by metal layer 3 causes interference with succeeding incident electromagnetic wave 111 passing through metal layer 2. Reflected electromagnetic wave 112 includes reflected electromagnetic waves 112a and 112b. Reflected electromagnetic wave 112a is reflected by upper surface 3A of metal layer 3, passes through metal layer 2, and reaches an observing point, as shown in FIG. 4A. Reflected electromagnetic wave 112b is reflected by upper surface 3A of metal layer 3, reflected by lower surface 2B of metal layer 2, and then, reflected again by metal layer 3 to reach detector 12 through metal layer 2, as shown in FIG. 4B. Reflected electromagnetic waves 112a and 112b interfere with incident electromagnetic wave 111. Optical path difference δ between incident electromagnetic wave 111 and reflected electromagnetic wave 112 is determined by a thickness d of hollow area 4, effective refractive index n in hollow area 4, and incidence angle θ that is an angle of incident electromagnetic wave 111 to a normal line perpendicular to the upper surface of metal layer 2, and is expressed as Formula 1.

δ=2×n×d×cos θ (Formula 1)

In the case that optical path difference δ is (m+½) times of a half of the wavelength of electromagnetic wave 111 in which is an integer not smaller than zero, reflected electromagnetic waves 112a and 112b are mutually cancelled out and are observed as a resonance absorption. In other words, wavelength λ satisfying Formula 2 disables detector 12 to observe reflected electromagnetic wave 112. This is fundamentally a multiple-reflection interference which is the same phenomena as Fabry-Perot interference.

2×n×d×cos θ=(m+½)×λ (Formula 2)

In above explanation, an interference between reflected electromagnetic wave 112a which entering into optical sensor 1, reflected once by upper surface 3A of metal layer 3, and reaches detector 12 and reflected electromagnetic wave 112b reflected twice by metal layer 2 and metal layer 3 and reaches detector 12 is described. This operation can be applied to a combination of reflected electromagnetic wave 112a and reflected electromagnetic wave 112b which reach the observing point after repeating reflections by a different odd number.

As clearly derived from Formula 2, the wavelength of incident electromagnetic wave 111 which causes the interference in hollow area 4 depends on the refractive index n of hollow area 4. Hence, a condition for interference in which the reflected light becomes invisible at detector 12 changes in response to a change of the effective refractive index in hollow area 4.

In the description below, for a simple argument and a least erroneous usage of optical sensor 1, it is assumed that incident electromagnetic wave 111 enters vertically to the upper surface of metal layer 2 from above optical sensor 1. Namely, angle θ in Formulas 1 and 2 is 0°. When incident electromagnetic wave 111 enters at an angle θ other than 0° or when detector 12 is placed in a different angle, calculation of Formula 2 is made with angle θ.

Metal layer 3 has a thickness not smaller than 100 nm. Incident electromagnetic wave 111 entering into upper surface 3A of metal layer 3 especially having a wavelength longer than about 550 nm is strongly reflected by a phenomenon so-called anomalous reflection of gold. Thickness t2 of metal layer 2 is small to pass incident electromagnetic wave 111 through metal layer 2. Metal layer 2 has a smaller reflectivity than metal layer 3.

For example, regarding interference between electromagnetic wave 112a reflected repetitively by k times and electromagnetic wave 112b reflected repetitively by (k+2) times, the electromagnetic wave reflected by lower surface 2B of metal layer 2 (k+2) times loses intensity more than the electromagnetic wave reflected k times. As a result, even if satisfying the condition of interference of Formula 2, reflected electromagnetic waves 112a and 112b do not fully cancel each other, and reduce the selectivity of an interfering wavelength, thereby making a resonance absorption peak wide and shallow.

Conventional optical sensor 201 disclosed in PTL 2 detects a change in the resonance absorption wavelength caused by a change in the refractive index of hollow area 204, thereby sensing whether trapper 207 is specifically bound to analyte 208A or not. In order to increase the sensitivity of optical sensor 201, it is necessary to identify a subtle change in the resonance absorption wavelength. For this reason, the resonance absorption peak is required to be sharp, and metal layer 202 is required to be thick as much as possible as long as permeability of electromagnetic wave 209A is maintained.

On the contrary, metal layer 2 of optical sensor 1 according to the embodiment has extremely small thickness t2 ranging from 5 nm to 30 nm for the following reason.

In order to find an optimal thickness of metal layer 2, plural samples having various thicknesses t2 are prepared, and then a change in a reflection spectrum is measured. Metal layer 2 and metal layer 3 are both made of an evaporated gold film. Metal layer 3 is has a thickness of 100 nm. Thickness d of hollow area 4, a distance between lower surface 2B of metal layer 2 and upper surface 3A of metal layer 3 in Formulas 1 and 2 is 840 nm.

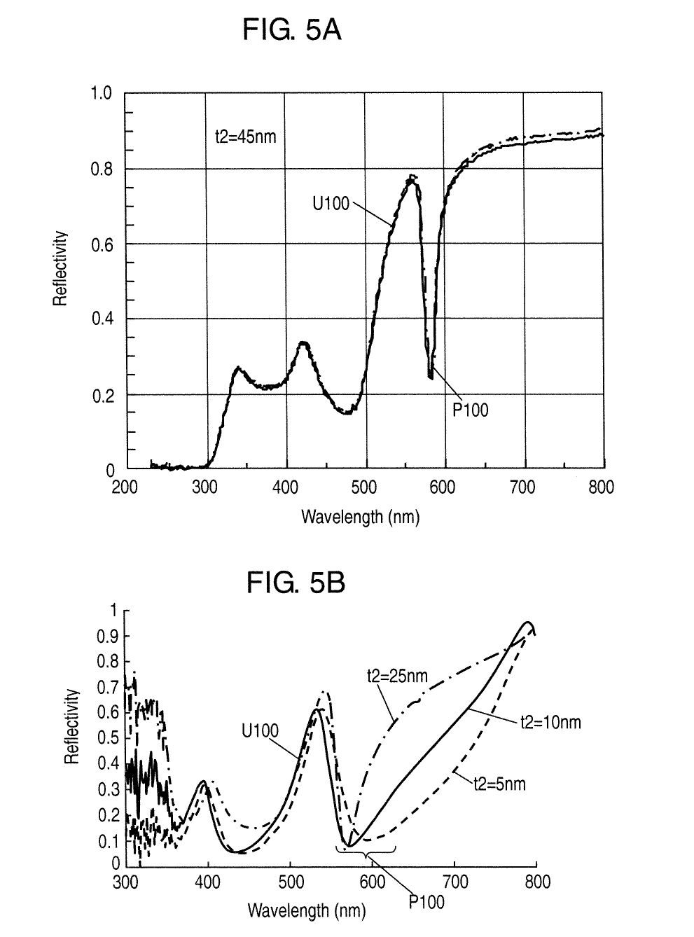

FIG. 5A shows a change in the reflection spectrum of a comparative example of an optical sensor in which thickness t2 of metal layer 2 is 45 nm. FIG. 5B shows a change in the reflection spectrum of metal layer 2 of optical sensor 1 according to the embodiment. As shown in FIG. 5A, the comparative example of the optical sensor, the reflectivity increases at portion U100 of wavelength longer than about 500 nm due to the anomalous reflection of gold. Sharp peak P100 of resonance absorption due to an optical resonance (Fabry-Perot interference phenomena) appears at about a wavelength of 590 nm. Color of the reflected light at this moment is gold, which is almost identical to a reflection color of gold.

As thickness t2 of metal layer 2 becomes smaller, the color of the reflected light becomes clearly different from gold color in visible observation, and becomes fresh green. As shown in FIG. 5B, as thickness t2 of metal layer 2 decreases, although the color of the reflected light (reflected electromagnetic wave 112) does not significantly change at a portion of the wavelength of the light (electromagnetic wave) shorter than about 500 nm, the shapes at a wavelength longer than resonance absorption peak P100 of about 590 nm are signficantly different from each other.

Resonance absorption peak P100 at about a wavelength of 590 nm is widened not simply in accordance with the wavelength selectivity as suggested above, but the reflectivity is reduced largely at wavelengths longer than 590 nm, and the resonance absorption peak is widened asymmetrically. This shape reduces reflection at a range of orange color to red color, and allows a pseudo peak structure having a peak at about a wavelength of 550 nm to appear. The pseudo peak structure is provided between a portion around the wavelength of 550 nm in which the reflectivity increases due to the anomalous reflection of gold and the resonance absorption peak P100 at about 590 nm due to the resonance absorption. This pseudo peak structure causes reflected light (reflected electromagnetic wave 112) to exhibit a bright green color.

Optical sensor 1 according to the embodiment utilizes the change in color caused by the pseudo peak structure as an indicator to detect the change in the effective reflective index in hollow area 4.

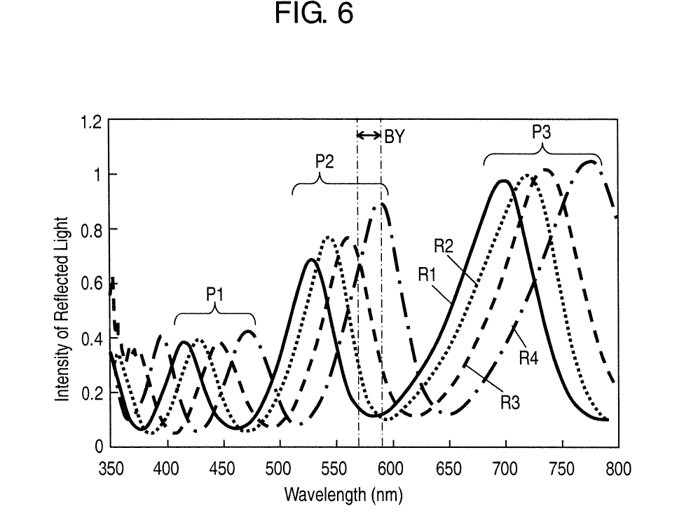

FIG. 6 shows a relation between a wavelength of the peak of the pseudo peak structure and a refractive index of hollow area 4 which is obtained by inputting reference solutions having known refractive indexes into hollow area 4 of optical sensor 1. The reference solutions are pure water having a refractive index of 1.33, isooctane having a refractive index of 1.39, cyclohexane having a refractive index of 1.426, and toluene having a refractive index of 1.497. FIG. 6 shows reflectivity R1 corresponding to the reference solution of pure water, reflectivity R2 corresponding to the reference solution of isooctane, reflectivity R3 corresponding to the reference solution of cyclohexane, and reflectivity R4 corresponding to the reference solution of toluene.

As shown in FIG. 6, each of reflectivities R1 to R4 has pseudo peak structures P1, P2 and P3. Each of center wavelengths of pseudo peak structures P1 to P3 shifts toward a longer wavelength as the refractive index becomes higher.

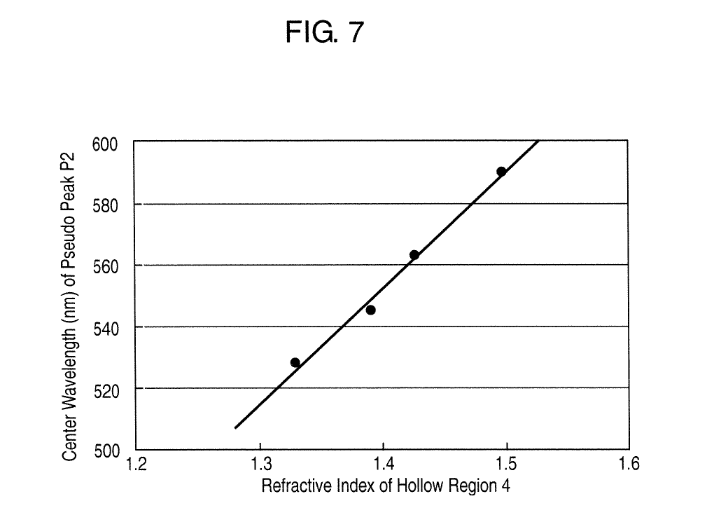

FIG. 7 shows a relation between a center wavelength of the pseudo peak structure P2 and a refractive index in hollow area 4 of optical sensor 1. As shown in FIG, 7, a change in the center wavelength with regard to the refractive index is approximated by a straight line. Thus, not only the resonance absorption peak but also the center wavelength in the pseudo peak structure formed between the resonance absorptions changes in accordance with the change in the refractive index of hollow area 4.

In conventional optical sensor 201, a change in color tone of the spectrum of reflected light of gold due to the losing of a narrow wavelength range of resonance absorption peak is detected. For example, a slight change in color of the reflected color of gold from reddish gold to greenish gold is detected. Thus, the change in the reflacitive index is not easily detected. In optical sensor 1 according to the embodiment, however, the pseudo peak structure is used as a reference of detecting, and reflective color in each refractive index is close to single color. This configuration allows the change in the refractive index to be easily determined.

As described above, in optical sensor 1 according to the embodiment, metal layer 2 is thin, and metal layers 2 and 3 are made of gold to reduce the selectivity of wavelength for the resonance absorption by interference. This configuration provides pseudo peak structures P1 to P3 which are not appeared in conventional optical sensor 201. Pseudo peak structures P1 to P3 allows optical sensor 1 to determine the change in the refractive index in hollow area 4 easier than conventional optical sensor 201.

A method of manufacturing optical sensor 1 according to the embodiment will be described below. The method of manufacturing optical sensor 1 includes at least the following three steps.

At the first step, an optical sensor including metal layer 2, metal layer 3, and hollow area 4 is prepared. Metal layer 2 have upper surface 2A and lower surface 2B, and is configured to have incident electromagnetic wave 111 supplied thereto. Metal layer 2 is made of gold and has a thickness not smaller than 5 nm and not larger than 30 nm. Metal layer 3 is made of gold and has upper surface 3A facing lower surface 2B of metal layer 2. Metal layer 2 and metal layer 3 may be jointed with a spacer, such as a pillar or a wall, as to maintain hollow area 4 effectively.

At the second step, a solute containing component 10 is input into hollow 4 by capillary phenomena.

At the third step, the solute input into hollow area 4 is dried by, e.g. vacuum drying. Then, composite bodies 10 are dispersed and disposed at at least one of an under part of metal layer 2 and an upper part of metal layer 3.

In optional sensor 1 according to the embodiment, it is not necessary to fix trapper 7 to an inside of hollow area 4 by chemical absorption. Trappers 7 may be input into hollow area 4 by a simple method as mentioned above, enhancing manufacturing efficiency of optical sensor 1.

Hollow area 4 may be provided at almost entire area between metal layer 2 and metal layer 3. The area includes an area where trappers 7 are not disposed.

Hollow area 4 may be formed in an area other than an area where the pillar and the wall supporting metal layer 2 and metal layer 3 are formed between metal layer 2 and metal layer 3. This area includes an area where trapper 7 is not disposed.

A corrosion prevention layer may be applied onto lower surface 2B of metal layer 2 and upper surface 3A of metal layer 3. In this case, hollow area 4 may be formed in an area other than where the corrosion prevention layer is formed between metal layer 2 and metal layer 3. This area does not include an area where surface trappers 7 are disposed on the surfaces of metal layer 2 and metal layer 3 which the corrosion prevention is not applied to.

Hollow area 4 is an area configured to have specimen 8 input thereto, and is secured in a part between metal layer 2 and metal layer 3.

The distance between metal layer 2 and metal layer 3 preferably ranges from 400 nm to 1600 nm. This distance allows analyte 8A to be specifically bonded to trapper 7, and allows pseudo peak structure P2 to shift across a wavelength range BY of yellow ranging from 570 nm to 590 nm between before and after the change of the refractive index of hollow area 4. At this moment, the reflected color changes from green to yellow or to orange in a categorical color different from green, so that the change in the refractive index can be easily identified visibly. The distance between metal layer 2 and metal layer 3 may more preferably range from 400 nm to 1000 nm.

In optical sensor 1 according to this embodiment, the center wavelength of the pseudo peak structure P2 is determined such that the center wavelength essentially shifts across the wavelength band BY of yellow before and after aggregate 10A is formed with composite bodies 10 and changes the refractive index of hollow area 4.

When analyte 8A exists in specimen 8, analyte 8A and trapper 7 form the agglomeration in hollow area 4, or composite body 10 and the agglomeration aggregate form aggregate 10A. Resultantly, the refractive index in hollow area 4 changes. The center wavelength of the pseudo peak structure P2 shifts substantially across the bandwidth of 570 nm to 590 nm (yellow wavelength band BY) before and after the change of the refractive index. More specifically, the peak wavelength before the change is shorter than 570 nm namely in the green categorical color zone shifts to a wavelength longer than 570 nm namely in the yellow or an orange categorical color zone after the change.

The peak wavelength after the change is preferably longer than 580 nm, which is the center of the yellow wavelength band BY.

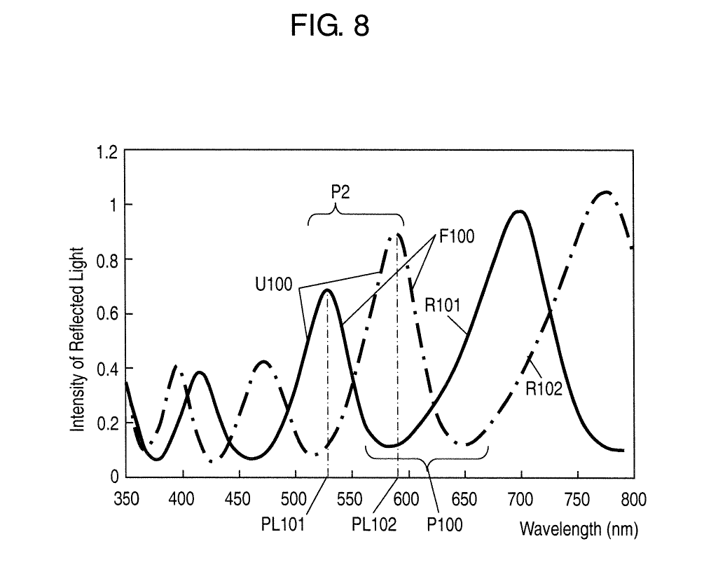

FIG. 8 shows a change in a spectrum of the reflected light of optical sensor 1 according to the embodiment. The peak spectrum structure before the refractive index is changed namely before trapper 7 and analyte 8A are bound is made up of (1) a reflectivity rising part where the reflectivity of the spectrum of the light reflected by gold making up metal layer 2 and metal 3 rises and (2) a part of resonance absorption peak P100 where the light reflected by metal layer 2 and metal layer 3 is superimposed on a spectrum and absorbed by interference under a condition the refractive index of hollow area 4 is still low. The center wavelength of pseudo peak structure P2 having such spectrum structure is first center wavelength PL101.

Similarly, the peak spectrum structure after trapper 7 and analyte 8A are bound (after the change) is made up of (1) a reflectivity rising part where the spectrum of light reflected by gold making up metal layers 2 and 3 rises and (2) a part of resonance absorption peak P100 where the light reflected by metal layer 2 and metal layer 3 is superimposed on an absorption spectrum and absorbed by interference under a condition that the refractive index of hollow area 4 become high. The center wavelength of this pseudo peak structure P2 having such spectrum structure is second center wavelength PL102.

As mentioned, the spectrum of the light reflected by metal layers 2 and 3 has the pseudo peak structure composed of part U100 where the spectrum rises to a local maximum value due to the reflection of gold of metal layers 2 and 3; and part F100 where the spectrum falls down from the local maximum value due to absorption by interference of the light reflected by metal layers 2 and 3.

First center wavelength PL101 is shorter than 570 nm, and second center wavelength PL102 is longer than 570 nm.

More preferably, first center wavelength PL101 is shorter than 580 nm, and second center wavelength PL102 is longer than 580 nm while at least one of a condition that first center wavelength PL101 is shorter than 570 nm and a condition that second center wavelength PL102 is longer than 590 nm is satisfied.

For human eyes, visible color is perceived successively changing from purple, an end of short wavelength, via blue, green, and yellow to red as the wavelength increases. When optical sensor 1 according to the embodiment senses existence or nonexistence of analyte 8A based on the change in color defined by the spectrum of the reflected light, it is important how large can be a changing amount in perception against a certain real amount of change in wavelength.

Upon perceiving colors, human eyes perceive a group of color including similar colors, not perceiving a ratio of output from three kinds of cone photoreceptor cell corresponding to red, green and blue. For instance, upon perceiving red color, human eyes perceive a category (categorical color) of red including various red ranging from dark red to orange red. It is called a categorical color perception. Therefore, human eyes easily determine colors as long as each belongs to a different color category even if the colors are on a continued color spectrum.

The categorical color distinguished by the perception of categorical color is studied from a linguistic cultural aspect. The reason is that the color which is not expressed as a word cannot be a categorical color. Red, yellow, green, blue, brown, pink, orange, white, gray and black are defined as a fundamental categorical color common to various languages.

For example, a light source of mono color having a very narrow line width in a different categorical color. The categorical color changes from blue to green, yellow, orange and to red as the wavelength is shifted from a short wavelength to a long wavelength. But a width of the wavelength corresponding to each color category is not uniform. From blue to green, the wavelength gradually changes from about 400 nm to about 570 nm. But, three different color categories, green, yellow and orange are perceived when the wavelength shifts by a narrow band width of 20 nm from 570 nm to 590 nm. This narrow band of only 20 nm from 570 nm to 590 nm is perceived as yellow.

That is, when the center wavelength of the pseudo peak structure P2 changes by shifting across the wavelength band BY of yellow, the categorical color changes even if the wavelength changes by only 20 nm, namely the categorical color changes from green to orange. The change in this wavelength band is visually identified more easily than a change in other wavelength band.

The wavelength at the local maximum value of the pseudo peak structure P2 is determined substantially by a thickness d of the hollow area, the distance between lower surface 2B of metal layer 2 and upper surface 3A of metal layer 3.

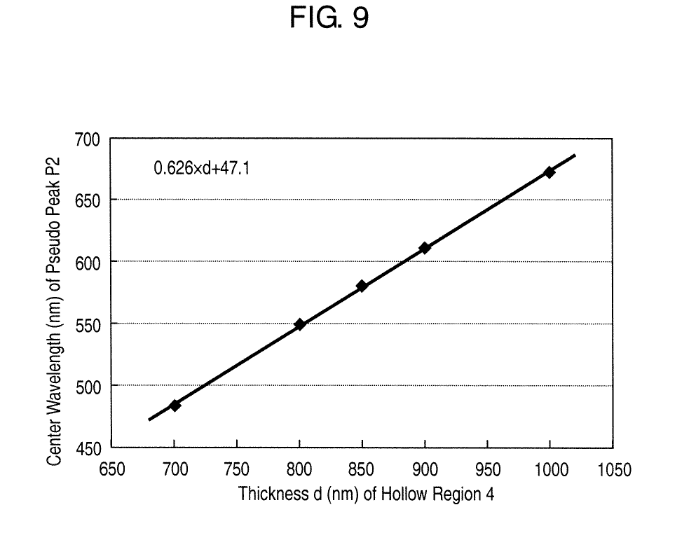

FIG. 9 shows a change in the center wavelength (wavelength at the local maximum value) of the pseudo peak structure P2 against a change in thickness d of hollow area 4 between metal layers 2 and 3. The refractive index of hollow area 4 is that of pure water. As shown in FIG. 9, the center wavelength of the pseudo peak structure P2 (wavelength at the local maximum value) changes along a straight line as thickness d of hollow area 4 changes.

In optical sensor 1 according to the embodiment, thickness d of hollow area 4, a distance between metal layers 2 and 3, is 820 nm according to the result of FIG. 9, so that the center wavelength of pseudo peak P2 becomes 560 nm when hollow area 4 is filled with pure water having a refractive index of 1.334. In this configuration, when the refractive index of hollow area 4 changes from that of pure water to that of isooctane having a refractive index of 1.39, the center wavelength of the pseudo peak P2 shifts from 560 nm to 590 nm. The change of the wavelength changes color of the reflected light from a green categorical color to an orange categorical color. In the case that a polystyrene latex bead is used as particle 9, the above change in the wavelength occurs when a change in the effective refractive index due to an aggregation rate of 40% occurs in hollow area 4.

Regarding thickness d of hollow area 4, i.e., the distance between metal layer 2 and metal layer 3 which determines the center wavelength of the pseudo peak structure P2, an optimum value of thickness d changes depending on an amount of change in the refractive index caused by the aggregation of composite bodies 10 and an absolute value of the refractive index in hollow area 4.

Since yellow wavelength band BY has a width of 20 nm, in order for the center wavelength of the pseudo peak structure P2 shifts across the yellow wavelength band BY before and after the aggregation, the change in the center wavelength or the difference between first center wavelength PL101 and second center wavelength PL102 is preferably not smaller than 10 nm.

When the amount of the change in the center wavelength is small, first center wavelength PL101 before reaction of trapper 7 and analyte 8A belongs to the green categorical color zone and the wavelength is long as much as possible. For this reason, it is necessary to strictly control the distance between metal layers 2 and 3 or thickness d of hollow area 4.

From a viewpoint of the categorical color, the change from green to yellow can be easily detected than the change from yellow to orange.

Therefore, in the case that the change amount in the center wavelength of pseudo peak structure P2 is not sufficiently large, first center wavelength PL101 before the reaction is preferably not longer than 560 nm around the longest wavelength of green color, and second center wavelength PL102 after the reaction is preferably longer than 560 nm. In this case, although the center wavelength of the pseudo peak structure P2 does not shift substantially across yellow wavelength band BY before and after the reaction, the categorical color changes from green to yellow, so that the change in color can be detected sensitively.

According to the embodiment, the refractive index of particle 9 determining the refractive index of hollow area 4 is larger than the refractive index of solvent 8C, so that the refractive index of hollow area 4 may increases when the aggregation of composite bodies 10 occurs.

The refractive index of particle 9 may be smaller than the refractive index of solvent 8C. In this case, thickness d of hollow area 4, the distance between metal layers 2 and 3 is determined such that the center wavelength of the pseudo peak structure before the reaction belongs to the categorical color of yellow or orange and that, after the reaction, belongs to the categorical color of green, thereby allowing the change in color to be easily detected.

In the exemplary embodiment, terms, such as “upper surface”, “lower surface”, “upper part” and “under part”, indicating directions indicate relative directions depending only on relationships of constituent components, such as metal layers 2 and 3, of optical sensor 1, and do not indicate absolute directions, such as a vertical direction.

### INDUSTRIAL APPLICABILITY

An optical sensor according to the invention has a small size and a simple structure, and is useful for a small and inexpensive biosensor and a chemical sensor.

### REFERENCE MARKS IN THE DRAWINGS

1 Optical Sensor
2 Metal Layer (First Metal Layer)
3 Metal Layer (Second Metal Layer)
4 Hollow Area
5 Holder
6 Holder
7 Trapper
8 Specimen
8A Analyte (Object Substance)
8B Other Solute
8C Solvent
9 Particle
10 Composite Body
11 Electromagnetic Source (Light Source)
12 Detector
111 Incident Electromagnetic Wave
112 Reflected Electromagnetic Wave

1. An optical sensor configured to be used with a plurality of trappers specifically bound to an object substance to detect whether the object substance exists or not in a specimen, the optical sensor comprising:
a first metal layer made of gold having a lower surface and an upper surface which is configured to have an electromagnetic wave supplied thereto; and
a second layer made of gold having an upper surface facing the lower surface of the first metal layer.
wherein a hollow area configured to be filled with the specimen is provided between the first metal layer and the second metal layer,
wherein the plurality of trappers are physically bonded to at least one of a lower side of the first metal layer and an upper side of the second metal layer, and
wherein a thickness of the first metal layer is not smaller than 5 nm and not larger than 30 nm.

2. The optical sensor according to claim 1, wherein a distance between the first metal layer and the second metal layer is not smaller than 400 nm and not larger than 1600 nm.

3. The optical sensor according to claim 2, wherein the distance between the first metal layer and the second metal layer is not smaller than 400 nm and not larger than 1000 nm.

4. The optical sensor according to claim 1,
wherein a spectrum of light reflected by the first metal layer and the second metal layer has a pseudo peak structure composed of: a part where the spectrum of the light rises to a local maximum value as a wavelength of the light increases due to reflection by gold of the first metal layer and the second metal layer; and a part where the spectrum of the light decreases from the local maximum value due to an absorption by interference of the light reflected by the first metal layer and the second metal layer, and
wherein it is determined whether or not the object substance exists in the specimen based on: a first wavelength at which the pseudo peak structure has the local maximum value while the trappers are not bonded to the object substance; and a second wavelength at which the pseudo peak structure has the local maximum value while the trappers are bonded to the object substance.

5. The optical sensor according to claim 4,
wherein the first wavelength is shorter than 580 nm and the second wavelength is longer than 580 nm, and
wherein at least one of a condition that the first wavelength is shorter than 570 nm and a condition that the second wavelength is longer than 590 nm is satisfied.

6. The optical sensor according to claim 4, wherein the first wavelength is shorter than 570 nm and the second wavelength is longer than 570 nm.

7. The optical sensor according to claim 1, wherein the plurality of the trappers is affixed to a particle.

8. A method of detecting an object substance in a specimen, comprising:
providing an optical sensor which include
a first metal layer made of gold having a lower surface and an upper surface which is configured to have an electromagnetic wave supplied thereto, and
a second layer made of gold having an upper surface facing the lower surface of the first metal layer, wherein a hollow area is provided between the first metal layer and the second metal layer, and a plurality of trappers to be configured to be specifically bound to the object substance are physically bonded to at least one of a lower side of the first metal layer and an upper side of the second metal layer; and
inputting the specimen into the hollow area by a capillary phenomenon.

9. The method according to claim 8, wherein the plurality of trappers are fixed to a particle.

10. A method of manufacturing an optical sensor, the method comprising:
providing an optical sensor which includes
a first metal layer made of gold having a thickness not smaller than 5 nm and not larger than 30 nm and having a lower surface and an upper surface which is configured to have an electromagnetic wave supplied thereto, and
a second layer made of gold having an upper surface facing the lower surface of the first metal layer, wherein a hollow area is provided between the first metal layer and the second metal layer;
inputting, by a capillary phenomenon, a plurality of solutes containing trappers to be specifically bound into an object substance; and
disposing the trappers at least one of a lower side of the first metal layer and an upper side of the second metal layer by drying the plurality of solutes after said inputting of the trappers into the hollow area.

11. The method according to claim 10, wherein the plurality of trappers are fixed to a particle.
